(19) United States
(12) Patent Application Publication
(10) Pub. No.: US 2013/0137917 A1
(43) Pub. Date: May 30, 2013

(54) Double emulsion core-shell nano-structure and preparation methods thereof

(71) Applicants: Bang-Jie Liao, Changhua County (TW); Shang-Hsiu Hu, Taoyuan (TW); San-Yuan Chen, Hsinchu City (TW)

(72) Inventors: Bang-Jie Liao, Changhua County (TW); Shang-Hsiu Hu, Taoyuan (TW); San-Yuan Chen, Hsinchu City (TW)

(73) Assignee: NATIONAL CHIAO TUNG UNIVERSITY, Hsinchu City (TW)

(21) Appl. No.: 13/385,296

(22) Filed: Feb. 13, 2012

(30) Foreign Application Priority Data
Nov. 29, 2011 (TW) 100143761

(51) Int. Cl. A61N 2/00; A61K 9/14; B82Y 5/00

(52) U.S. Cl. 600/9; 424/400; 977/838; 977/906

(57) ABSTRACT

A double-emulsion core-shell nano-structure and preparation methods thereof is provided. The double-emulsion core-shell nano-structure is a structure of an oil shell enclosing a water core. The double-emulsion core-shell nano-structure can be prepared by simply mixing and stirring to emulsify an aqueous solution of a water soluble polymer and an organic solution of hydrophobic paramagnetic nanoparticles.

### CROSS-REFERENCE TO RELATED APPLICATION

This application claims the priority benefit of Taiwan application serial no. 100143761, filed Nov. 29, 2011, the full disclosure of which is incorporated herein by reference.

### BACKGROUND

1. Technical Field

The disclosure relates to a nano-structure and a preparation method thereof. More particularly, the disclosure relates to a double-emulsion core-shell nano-structure and a preparation method thereof.

2. Description of Related Art

At present, core-shell nano-structures made by some organic materials have been used as drugs carrier to carry drugs. These core-shell nano-structures include liposomes or micelles formed by amphoteric polymers. However, these core-shell nano-structures usually have problems of unstable structure and complicated preparation methods. Thus, the preparing conditions are difficult to be controlled to form these core-shell nano-structures with enough stability.

### SUMMARY

In one aspect, the present invention is directed to a double-emulsion core-shell nano-structure, which can be prepared by a simple emulsifying method using a single step of mixing and stirring.

The double-emulsion core-shell nano-structure above comprises an aqueous core and an oily shell surrounding the aqueous core. A composition of the oily shell comprises a water-soluble polymer and a plurality of hydrophobic paramagnetic nanoparticles, without using a surfactant. The aqueous core can accommodate a hydrophilic drug, and the oily shell can accommodate a hydrophobic drug.

According to an embodiment of this invention, the water-soluble polymer is polyvinyl alcohol having a molecular weight of 3,000-130,000 or polyvinyl pyrrolidone having having a molecular weight of 400,000-2,800,000.

According to another embodiment of this invention, the hydrophobic paramagnetic nanoparticles including Fe₂O₃, Fe₃O₄, CoFe₂O₄ or MnFe₂O₄ nanoparticles are modified by hydrophobic functional groups thereon.

In another aspect, this invention directs to a method of preparing double-emulsion core-shell nano-structures.

According to an implementation way of this invention, a single emulsifying method for preparing the double-emulsion core-shell nano-structures is provided. In this method, an aqueous solution and an organic solution are respectively prepared first. The aqueous solution contains a water-soluble polymer, and the organic solution comprises hydrophobic paramagnetic nanoparticles. Then, the aqueous solution and the organic solution are mixed and stirred to form an emulsion solution contains the double-emulsion core-shell nano-structures above.

According to an embodiment of this invention, in the above single emulsifying method, a hydrophilic drug and a hydrophobic drug can be optionally added into the aqueous solution and the organic solution, respectively, such that the double-emulsion core-shell nano-structures can be carriers of the hydrophilic drug, the hydrophobic drug, or a combination thereof.

According to another implementation way of this invention, a double emulsifying method for preparing the double-emulsion core-shell nano-structures is provided. In this method, a first aqueous solution and an organic solution are respectively prepared first. The first aqueous solution contains a hydrophilic drug and a first water-soluble polymer, and the organic solution comprises hydrophobic paramagnetic nanoparticles. Then, the first aqueous solution and the organic solution are mixed and stirred to form a water-in-oil emulsion solution. Next, a second aqueous solution is prepared. The second aqueous solution contains a second water-soluble polymer. Finally, the water-in-oil emulsion solution and the second aqueous solution are mixed and stirred to form water-in-oil emulsion solution containing the double-emulsion core-shell nano-structures above.

According to an embodiment of this invention, in the above double emulsifying method, a hydrophobic drug can be further added into the organic solution to form the double-emulsion core-shell nano-structures carrying both the hydrophilic and the hydrophobic drugs.

According to another embodiment, in the above single and double emulsifying methods, the hydrophobic paramagnetic nanoparticles including Fe₂O₃, Fe₃O₄, CoFe₂O₄ or MnFe₂O₄ nanoparticles are modified by hydrophobic functional groups thereon.

According to yet another embodiment, in the above single and double emulsifying methods, the method of the mixing and stirring step comprises ultrasound sonication.

According to yet another embodiment, in the above single and double emulsifying methods, a step of removing the organic solvent can be further performed after obtaining the double-emulsion core-shell nano-structures. This can make the finally obtained double-emulsion core-shell nano-structures more suitable to be used in a living organism.

In light of foregoing, only using simple steps of mixing and stirring and without adding any surfactans, the double-emulsion core-shell nano-structures, which can serve as a drug carrier, can be obtained.

The above presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

Many of the attendant features will be more readily appreciated as the same becomes better understood by reference to the following detailed description considered in connection with the accompanying drawings.

### BRIEF DESCRIPTION OF THE DRAWINGS

FIG. 1A is a cross-sectional diagram of a double-emulsion core-shell nano-structure according to an embodiment of this invention.

FIG. 1B is a cross-sectional diagram of a double-emulsion core-shell nano-structure according to another embodiment of this invention.

FIG. 2A is a flow diagram of the single emulsifying method according to an embodiment of this invention.

FIG. 2B is a flow diagram of the double emulsifying method according to another embodiment of this invention.

FIGS. 3A and 3B are scanning electronic microscopic (SEM) and transmission electronic microscopic (TEM) images of the obtained double-emulsion core-shell nano-structures containing hydrophobic Paclitaxel.

FIGS. 4A-4D are particle diameter distribution results, analyzed by dynamic light scattering, of the double-emulsion core-shell nano-structures.

FIG. 5 is the analysis result obtained from measuring the IO-OA nanoparticles and the obtained double-emulsion core-shell nano-structures by superconducting quantum interference device.

FIG. 6 is the obtained spin-lattice relaxation time (T₁) and the spin-spin relaxation time (T₂), analyzed by MRI, of the double-emulsion core-shell nano-structures containing Paclitaxel.

FIGS. 7A-7B are controlled release curves of the double-emulsion core-shell nano-structures containing Paclitaxel.

FIG. 8 is a TEM image of the obtained double-emulsion core-shell nano-structures containing hydrophilic gold nano-rods.

FIG. 9 is a controlled release curve of the double-emulsion core-shell nano-structures containing vitamin B12.

FIG. 10 is natural release curves of Doxorubicin from the double-emulsion core-shell nano-structures containing Doxorubicin.

FIG. 11 is natural release curves of Doxorubicin and Paclitaxel from the double-emulsion core-shell nano-structures containing both Paclitaxel and Doxorubicin.

FIGS. 12A and 12B are controlled release curves of Doxorubicin and Paclitaxel from the double-emulsion core-shell nano-structures containing both hydrophobic Paclitaxel and hydrophilic Doxorubicin, respectively.

FIGS. 13A and 13B are natural release curves of Doxorubicin and Paclitaxel from the double-emulsion core-shell nano-structures containing both hydrophobic Paclitaxel and hydrophilic Doxorubicin, respectively.

FIGS. 14A and 14B are controlled release curves of Doxorubicin and Paclitaxel from the double-emulsion core-shell nano-structures containing both hydrophobic Paclitaxel and hydrophilic Doxorubicin, respectively.

FIGS. 15A and 15B are SEM and TEM images of the obtained double-emulsion core-shell nano-structures made by PVP.

FIGS. 16A and 16B are the cytocompatibility test results of the double-emulsion core-shell nano-structures with and without surface modification, respectively.

### DETAILED DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### Double-Emulsion Core-Shell Nano-structure

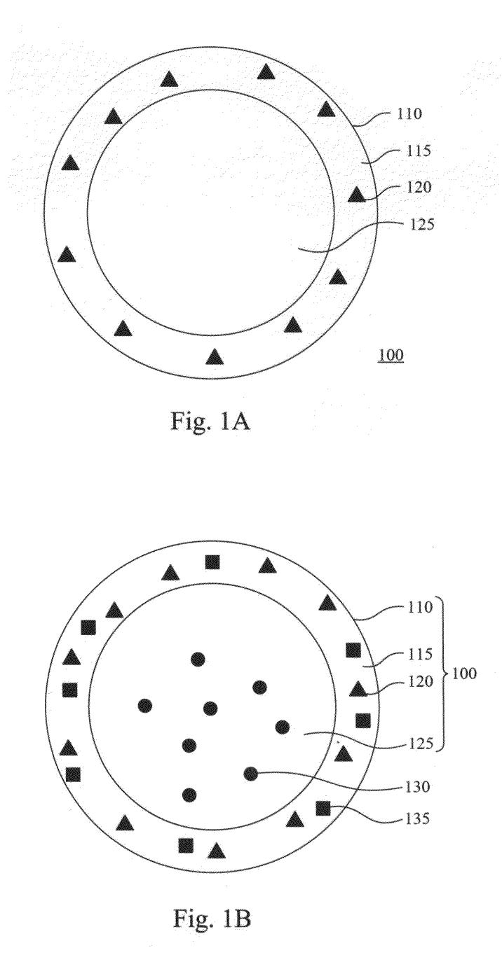

FIG. 1A is a cross-sectional diagram of a double-emulsion core-shell nano-structure according to an embodiment of this invention. In FIG. 1A, the double-emulsion core-shell nano-structure 100 is composed of an oily shell 110 surrounding an aqueous core 125. The oily shell comprises a water-soluble polymer 115 and hydrophobic paramagnetic nanoparticles 120.

The above water-soluble polymer 115 can be polyvinyl alcohol (PVA) or polyvinyl pyrrolidone (PVP). Generally, the hydrophilicity of a water-soluble polymer is related to its molecular weight, i.e., the chain length of the water-soluble polymer. Therefore, if the molecular weight of the water-soluble polymer is too low to make the solubility of the water-soluble polymer in water too high, the double-emulsion core-shell nano-structure 100 cannot be formed by the water-soluble polymer. However, if the molecular weight of the water-soluble polymer is too high to make the solubility of the water-soluble polymer in water too low, only solid polymer spheres can be formed by water-soluble polymer. Accordingly, in an embodiment, the molecular weight of the polyvinyl alcohol above is better to be 3,000-130,000, and even better to be 3,000-78,000. In another embodiment, the molecular weight of the polyvinyl pyrrolidone above is better to be 400,000-2,800,000, and even better to be 560,000-1,300,000.

Generally, one end of a surfactant molecule is hydrophilic, and the other end of the surfactant is hydrophobic. Therefore, only one oil-water interface can be stabilized by surfactant molecules, and a core-shell nano-structure is thus hard to be formed by surfactants. Unless the surfactants can form a lipid biilayer structure, then it can have an opportunity to form liposomes with core-shell structure by surfactants.

However, water-soluble polymers are different from surfactants. For example, polyvinyl alcohol has many hydrophilic hydroxyl groups. Since the molecular chain can rotate, each of the hydroxyl groups can be rotated toward either the aqueous core 125 or the aqueous solution outside the oily shell 110. Therefore, polyvinyl alcohol can simultaneously stabilize two oil-water interfaces to form double-emulsion core-shell nano-structure 100.

The hydrophobic paramagnetic nanoparticles 120 above can be Fe₂O₃, Fe₃O₄, CoFe₂O₄ or MnFe₂O₄ nanoparticles, which are modified by hydrophobic functional groups. The hydrophobic functional groups can be long-chained alkyl groups or long-chained alkenyl groups, such as oleic acid or oleylamine. The hydrophobic paramagnetic nanoparticles 120 can stabilize the oily shell 110 to prevent the oily shell 110 from collapsing. Besides being a contrast agent of magnetic resonance imaging (MRI), the hydrophobic paramagnetic nanoparticles 120 also can be used to locally heat and then break the oily shell 110 by magnetic fluid hyperthermia under an alternative magnetic field.

Since the double-emulsion core-shell nano-structure 100 has the oily shell 110 and the aqueous core 125 to respectively accommodate a hydrophobic drug and a hydrophilic drug therein, the double-emulsion core-shell nano-structure 100 can be used as a carrier of the hydrophobic drug, the hydrophilic drug, or a combination thereof. Furthermore, the release rate of a drug can be controlled by the strength and on/off state of an applied external alternative magnetic field.

FIG. 1B is a cross-sectional diagram of a double-emulsion core-shell nano-structure according to another embodiment of this invention. In FIG. 1B, a hydrophilic drug 130 is accommodated in the aqueous core 125 of the double-emulsion core-shell nano-structure 100. A hydrophobic drug 135 is accommodated in the oily shell 110 of the double-emulsion core-shell nano-structure 100.

### Method of Preparing Double-Emulsion Core-Shell Nano-Structure

The preparation method of double-emulsion core-shell nano-structure can be simply divided to single emulsifying method and double emulsifying method.

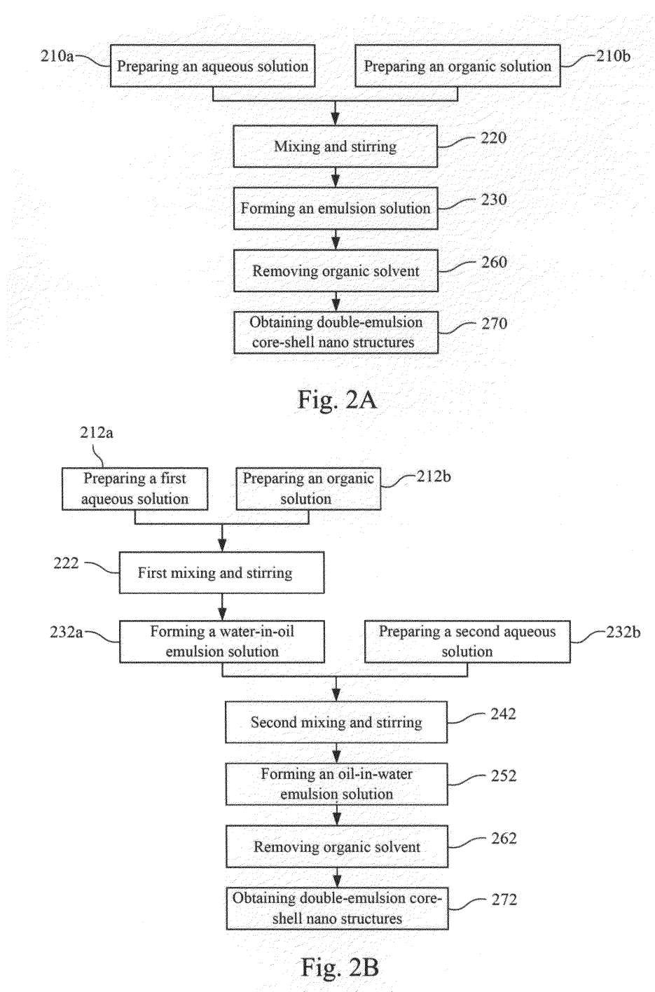

FIG. 2A is a flow diagram of the single emulsifying method according to an embodiment of this invention. The single emulsifying method is more suitable for preparing double-emulsion core-shell nano-structures containing only hydrophilic drugs or only hydrophobic drugs, but is not limited thereto. In FIG. 2A, an aqueous solution (step 210a) and an organic solution (step 210b) are respectively prepared. After mixing and stirring (step 220), an emulsion solution containing double-emulsion core-shell nano-structures are formed (step 230). Next, the organic solvent in the emulsion solution is removed (step 260) to obtain the double-emulsion core-shell nano-structures (step 270) suitably applied in living organisms.

The aqueous solution of the step 210a contains water-soluble polymer and water, and can optionally contain at least a hydrophilic drug. The organic solution of the step 210b contains hydrophobic paramagnetic nanoparticles and an organic solvent, and can optionally contain at least a hydrophobic drug. When the organic solution contains only hydrophobic paramagnetic nanoparticles, the organic solvent is better to have the properties of effectively dissolving or dispersing the hydrophobic paramagnetic nanoparticles, immiscible with water, and lower boiling point. When the organic solution further contain a hydrophobic drug, the organic solvent is better to further have the property of effectively dissolving or dispersing the hydrophobic drug. The reason for choosing an organic solvent with lower boiling point is that the organic solvent can be easily removed without heating to prevent the outer shape of the double-emulsion core-shell nano-structures to be influenced by non-controllable adverse effects. The boiling point of the organic solvent can be lower than 90° C. The organic solvent can be chloroform, dichloromethane, trichloroethane, or acetonitrile.

The method of mixing and stirring in the step 220 can be ultrasound sonication, for example. The method of removing the organic solvent in the step 260 can be volatilization or reduced pressure distillation, and the temperature of removing the organic solvent is better to be lower than 90° C.

FIG. 2B is a flow diagram of the double emulsifying method according to another embodiment of this invention. The double emulsifying method can increase the encapsulation efficiency of a hydrophilic drug, but is not limited thereto. In the single emulsifying method of FIG. 2A, only one emulsifying step is used to form oil-in-water emulsion solution. Therefore, besides the aqueous cores of the double-emulsion core-shell nano-structures, the hydrophilic drug can also distribute in the water phase outside the double-emulsion core-shell nano-structures. This makes the encapsulation efficiency of the hydrophilic drugs is lower. Hence, the double emulsifying method is used to increase the encapsulation efficiency of hydrophilic drugs.

In FIG. 2B, a first aqueous solution (step 212a) and an organic solvent (step 212b) are respectively prepared. A first mixing and stirring step (step 222) is performed to form a water-in-oil emulsion (step 232a) by using a small amount of the first aqueous solution and excess amount of the organic solution. This is the first emulsifying step.

The first aqueous solution of the step 212a contains at least a hydrophilic drug, a first water-soluble polymer, and water to facilitate the water drops in the water-in-oil emulsion solution completely enclosing the hydrophilic drug. The organic solution of the step 212b contains hydrophobic paramagnetic nanoparticles and an organic solvent, and can optionally contain at least a hydrophobic drug. The requirements for the organic solvent of step 212b in FIG. 2B are similar to those for the organic solvent of step 210b in FIG. 2A, and hence are omitted here.

Next, a second aqueous solution (step 232b) is prepared. A second mixing and stirring step (step 242) is performed to form an oil-in-water emulsion solution containing double-emulsion core-shell nano-structures (step 252) by using the water-in-oil emulsion solution and the second aqueous solution. This is the second emulsifying step. Since the double-emulsion core-shell nano-structures are formed in the second emulsifying step, the second aqueous solution is better to contain only a second water-soluble polymer and water to effectively and completely enclosing the hydrophilic drugs by double-emulsion core-shell nano-structures.

The first water-soluble polymer and the second water-soluble polymer can be the same polymer or different polymers, and the molecular weight of the first water-soluble polymer and the second water-soluble polymer can be the same or different (newly added sentences to support claim 15). The method of the first mixing and stirring step (step 222) and the second mixing and stirring step (step 242) can be ultrasound sonication, for example.

Finally, the organic solvent in the oil-in-water emulsion is removed (step 262) to obtain the double-emulsion core-shell nano-structures (step 272) suitably applied in living organisms. The method of removing the organic solvent in the step 262 can be volatilization or reduced pressure distillation.

### Embodiment 1

### Enclosing Hydrophobic Paclitaxel (PTX)

Double-emulsion core-shell nano-structures containing hydrophobic Paclitaxel (PTX) was prepared by following the process flow of FIG. 2A. An aqueous solution of 0.02 g/ml polyvinyl alcohol and an organic solution were respectively prepared. The molecular weights of the polyvinyl alcohol used above were 16,000, 47,000, and 61,000. The organic solvent used in the organic solution above was chloroform, and the organic solution contained 0.03 g/ml Paclitaxel and 0.02 g/ml Fe₃O₄ nanoparticles surface-modified by oily acid (abbreviated as IO-OA A nanoparticles below) with a diameter of about 5 nm. Please refer to Sun, S. H.; Zeng, H.; Robinson, D. B.; Raoux, S.; Rice, P. M.; Wang, S. X.; Li, G. X. Journal of the American Chemical Society, 2004, 126, (1), 273-279, which is incorporated herein by reference, for the preparation method of the IO-OA nanoparticles.

2.5 ml of the aqueous solution above and 1 ml of the organic solution above were mixed and then completely emulsified by 20 kHz ultrasound. Then, the chloroform was completely volatized at room temperature. Finally, the product of the double-emulsion core-shell nano-structures were washed by deionized water and isolated by 8500 rpm centrifugation, and then re-dispersed in deionized water.

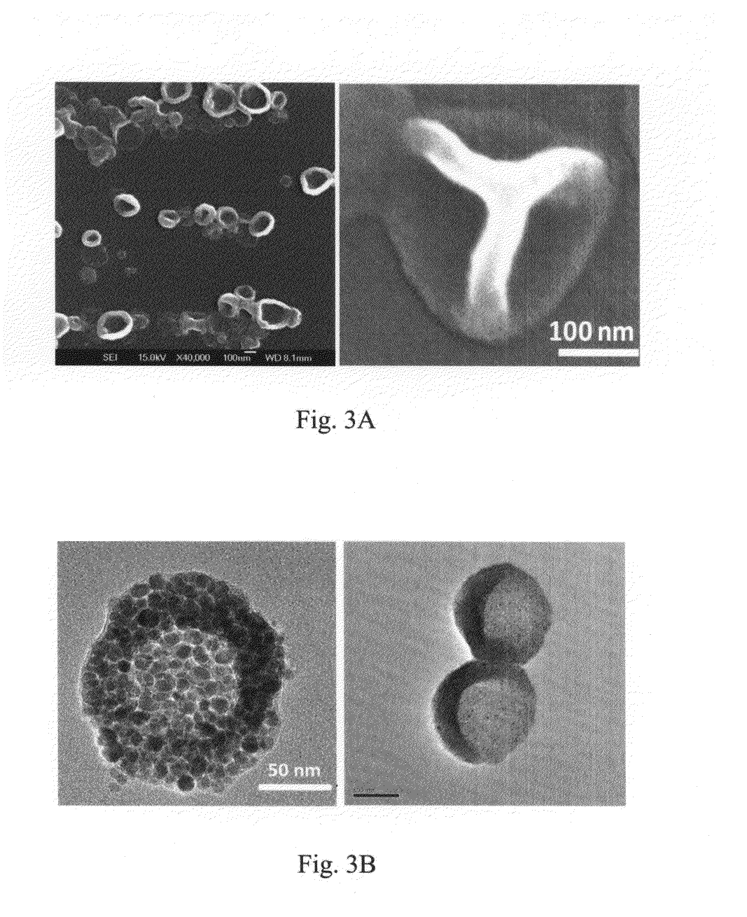

FIGS. 3A and 3B are scanning electronic microscopic and transmission electronic microscopic images of the obtained double-emulsion core-shell nano-structures containing Paclitaxel. Since the double-emulsion core-shell nano-structures in FIG. 3A had been vacuum-dried and the polymer chain of polyvinyl alcohol is soft, the oily shell were collapsed in a bowl shape. In FIG. 3B, it can be seen that the double-emulsion core-shell nano-structures were in a hollow sphere structure and IO-OA nanoparticles (darker color ones) were dispersed in the oily shells.

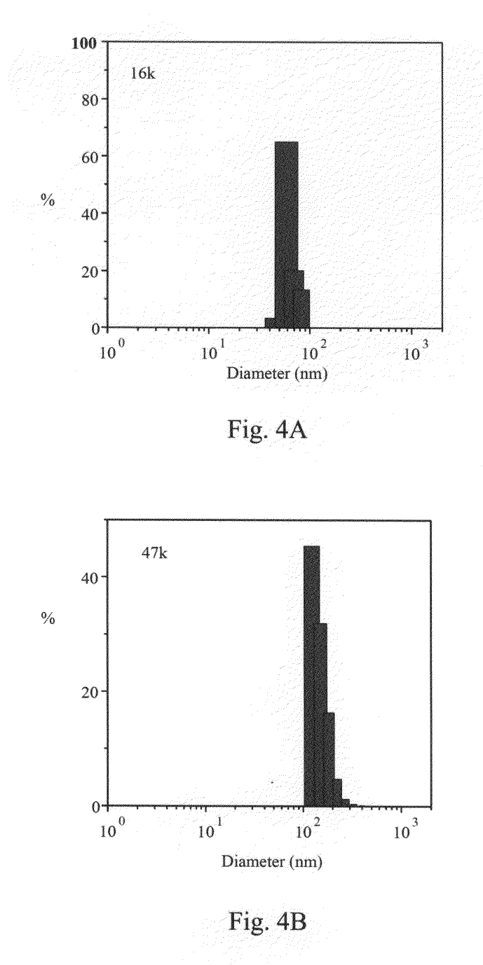

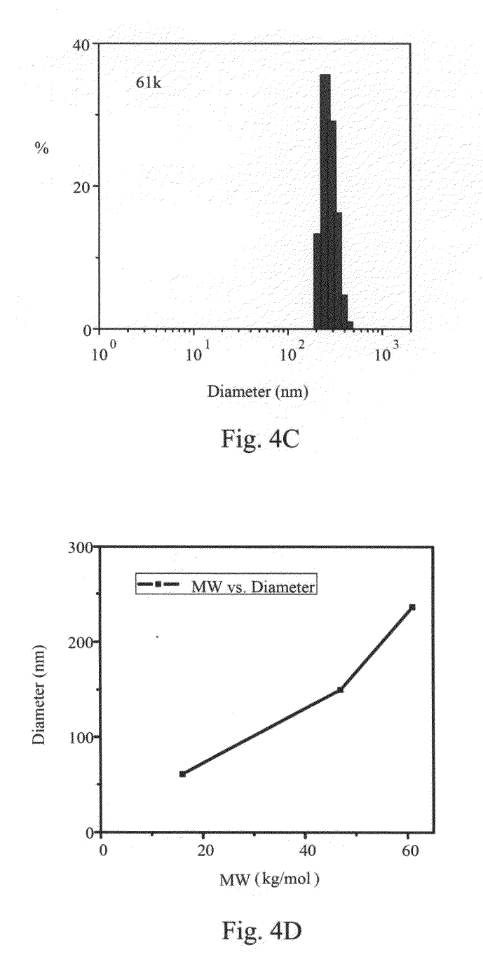

FIGS. 4A-4D are particle diameter distribution results, analyzed by dynamic light scattering, of the double-emulsion core-shell nano-structures. From FIGS. 4A-4D, it can be known that the particle diameter distributions were quite centralized for all polyvinyl alcohols with various molecular weights of 16,000 (FIG. 4A), 47,000 (FIG. 4B), and 61,000 (FIG. 4C). From FIG. 4D, it can be known that the diameter is greater when the molecular weight of the polyvinyl alcohol is greater.

Table 1 lists some related measured data about using polyvinyl alcohols with various molecular weights to prepare double-emulsion core-shell nano-structures containing Paclitaxel. In Table 1, the double-emulsion core-shell nano-structure is referred to as carrier, and the aqueous core is referred to as core.

TABLE 1
Using polyvinyl alcohols with various molecular weights to
prepare double-emulsion core-shell nano-structures containing Paclitaxel

| Molecular | Core | Carrier | PTX | PTX loading |
|---|---|---|---|---|
| weight of | diameter | diameter | Encapsulation | capacity |
| PVA | (nm) | (nm) | efficiency (%)* | (mg/g carrier) |
| 16k | 108 | 158 | 97 | 8.78 |
| 47k | 47 | 144 | 97 | 8.78 |
| 61k | 31 | 137 | 96 | 8.69 |

*Encapsulation efficiency (%) = PTX loading capacity/PTX total amount × 100%

From Table 1, it can be known that the hydrophilicity is poorer when the molecular weight of the polyvinyl alcohol was greater. Thus, the volume of the inner aqueous core was smaller, and the oily shell was thicker. However, the encapsulation efficiency and the loading capacity were not varied too much. The encapsulation efficiency for the double-emulsion core-shell nano-structure encapsulating Paclitaxel was more than 95%, which is quite great.

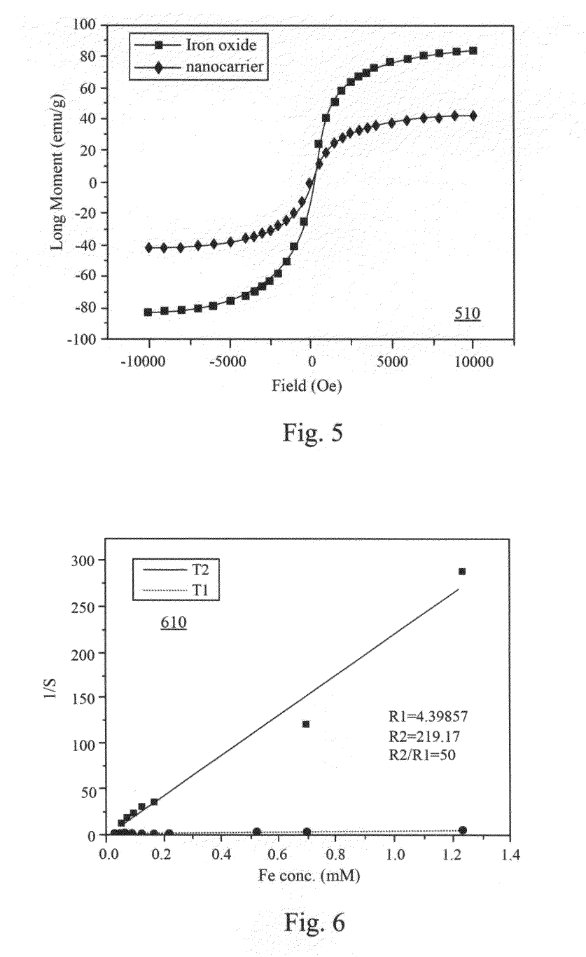

Next, some magnetic properties of the double-emulsion core-shell nano-structure containing Paclitaxel were tested. First, superconducting quantum interference device (SQUID) was used to analyze whether the IO-OA nanoparticles and the obtained double-emulsion core-shell nano-structures possessed paramagnetic property or not. The obtained result was shown in FIG. 5. In FIG. 5, since no magnetic hysteresis loops occurred for both the IO-OA nanoparticles and the obtained double-emulsion core-shell nano-structures, it can be known that both of them were paramagnetic.

Then, magnetic resonance image (MRI) was used to analyze the spin-lattice relaxation time (T₁) and the spin-spin relaxation time (T₂) of the double-emulsion core-shell nano-structures containing Paclitaxel. The obtained results were shown in FIG. 6. In FIG. 6, it can be seen that the spin-spin relaxation rate (R₂=1/T₂) was up to about 200, which was quite fast and 50 times of the spin-lattice relaxation rate (R₁=1/T₁). Therefore, the double-emulsion core-shell nano-structures are quite suitable to be used as contrast agent of magnetic resonance image.

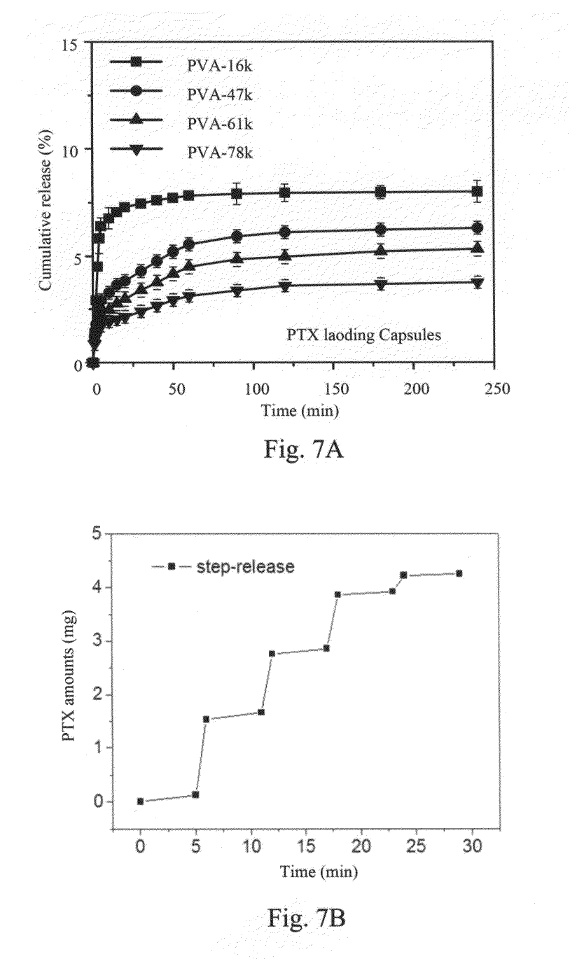

FIGS. 7A-7B are controlled release curves of the double-emulsion core-shell nano-structures containing Paclitaxel. From FIG. 7A, it can be known that the oily shell was thicker when the molecular weight of the polyvinyl alcohol was greater, and thus the aligned structure of the polyvinyl alcohol was more stable. Hence, the natural release rate of the Paclitaxel from the double-emulsion core-shell nano-structures is slower. From FIG. 7B, it can be known that the Paclitaxel release rate could be rapidly increased when a 200 Oe alternative magnetic field was turned on for 1 minute until the 200 Oe alternative magnetic field was turned off. Therefore, it can be known that an alternative field could be used to vibrate the IO-OA nanoparticles to locally heat the double-emulsion core-shell nano-structures. Then, the heated polyvinyl alcohol of the oily shell was dissolved to release Paclitaxel. Moreover, the stepwise heating did not break the double-emulsion core-shell nano-structures.

### Embodiment 2

### Enclosing Hydrophilic Gold Nano-Rods

Double-emulsion core-shell nano-structures containing hydrophilic gold nano-rods were prepared by following the process flow of FIG. 2A. An aqueous solution and an organic solution were respectively prepared. The aqueous solution above contained 0.02 g/ml polyvinyl alcohol (MW 16,000) and an excess amount of hydrophilic gold nano-rods having a length smaller than the diameter of the carriers. Please refer to Mitamura, K., Imae, T., Saito, N., and Takai, O., The Journal of Physical Chemistry B 111(30), 8891 (2007), which is incorporated herein by reference, for the preparation method of hydrophilic gold nano-rods. The organic solvent used in the organic solution above was chloroform, and the organic solution contained 0.02 g/ml IO-OA nanoparticles with diameters about 5 nm.

0.5 ml of the aqueous solution and 0.2 ml of the organic solution were mixed and then completely emulsified by 20 kHz supersonics. Next, the chloroform of the emulsion solution was completely volatized at room temperature. Finally, the product of the double-emulsion core-shell nano-structures were washed by deionized water and isolated by 8500 rpm centrifugation, and then re-dispersed in deionized water.

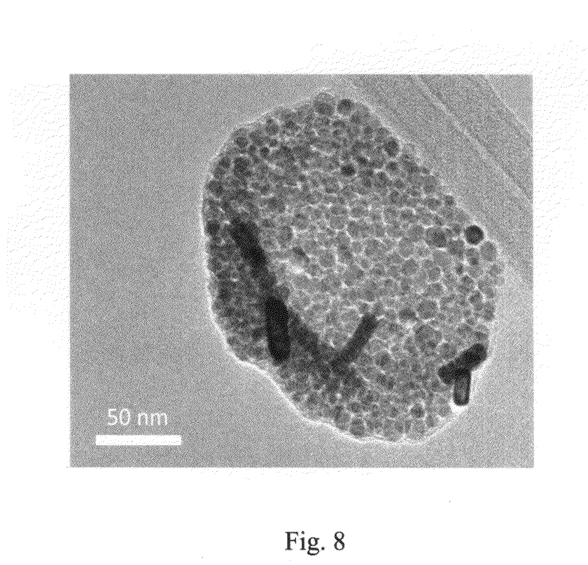

FIG. 8 is a TEM image of the obtained double-emulsion core-shell nano-structures containing hydrophilic gold nano-rods. From FIG. 8, it can be seen that the hydrophilic gold nano-rods (i.e., the shadow in rod shaped) were positioned in the aqueous cores of the double-emulsion core-shell nano-structures. Therefore, a hydrophilic material can be encapsulated in the aqueous cores of the double-emulsion core-shell nano-structures.

### Embodiment 3

### Enclosing Hydrophilic Vitamin B12

Double-emulsion core-shell nano-structures containing hydrophilic vitamin B12 were prepared by following the process flow of FIG. 2B. In the first emulsifying step, a first aqueous solution and an organic solution were respectively prepared. The first aqueous solution above contained 0.02 g/ml polyvinyl alcohol (MW 10,000-25,000) and 0.001 g/ml vitamin B12. The organic solvent used in the organic solution above was chloroform, and the organic solution contained 0.02 g/ml IO-OA nanoparticles with diameters about 5 nm. 0.2 ml of the aqueous solution and 0.5 ml of the organic solution were mixed and then completely emulsified by 20 kHz supersonics to form water-in-oil emulsion solution.

Next, the second emulsifying step was performed. A second aqueous solution containing 0.02 g/ml polyvinyl alcohol (MW 16,000) was prepared. The water-in-oil emulsion solution above and 1.75 ml of the second aqueous solution were mixed and then completely emulsified by 20 kHz supersonics to form a oil-in-water emulsion solution. Next, the chloroform of the oil-in-water emulsion solution was completely volatized at room temperature. Finally, the product of the double-emulsion core-shell nano-structures were washed by deionized water and isolated by 8500 rpm centrifugation, and then re-dispersed in deionized water.

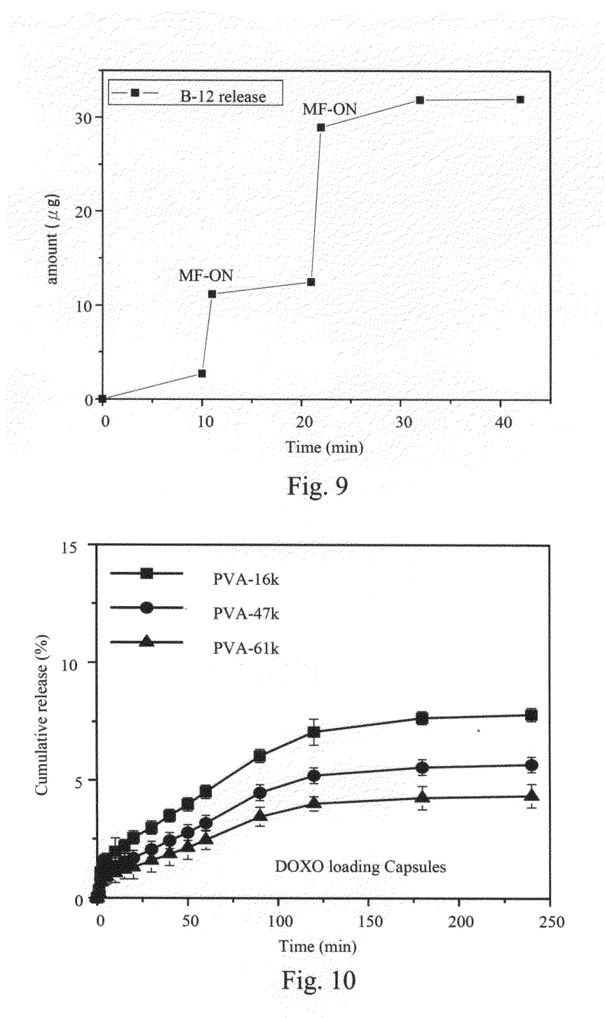

FIG. 9 is a controlled release curve of the double-emulsion core-shell nano-structures containing vitamin B12. In FIG. 9, a 200 Oe alternative magnetic field was turned on at the tenth minute and the twentieth minute for 1 minute. Therefore, it can be seen that there are two concentration fast-increased periods. After turning off the alternative magnetic field, only a small amount of vitamin B12 can be released.

### Embodiment 2

### Enclosing Hydrophilic Doxorubicin (DOXO)

Double-emulsion core-shell nano-structures containing hydrophilic

Doxorubicin were prepared by following the process flow of FIG. 2A. An aqueous solution and an organic solution were respectively prepared. The aqueous solution above contained 0.02 g/ml polyvinyl alcohol (MW 16,000, 47,000, and 61,000) and 0.002 g/ml Doxorubicin. The organic solvent used in the organic solution above was chloroform, and the organic solution contained 0.02 g/ml IO-OA nanoparticles with diameters about 5 nm.

0.2 ml of the aqueous solution and 0.5 ml of the organic solution were mixed and then completely emulsified by 20 kHz supersonics. Next, the chloroform of the emulsion solution was completely volatized at room temperature. Finally, the product of the double-emulsion core-shell nano-structures were washed by deionized water and isolated by 8500 rpm centrifugation, and then re-dispersed in deionized water.

The related data of the finally obtained double-emulsion core-shell nano-structures containing Doxorubicin are listed in Table 2. In Table 2, the double-emulsion core-shell nano-structure is referred to as carrier, and the aqueous core is referred to as core. From Table 2, it can be known that the hydrophilicity is poorer when the molecular weight of the polyvinyl alcohol is greater. Therefore, the volume of the aqueous cores is smaller, and the thickness of the oily shell is greater. The encapsulation efficiency and the loading capacity of the Doxorubicin were thus decreased.

TABLE 2
Using polyvinyl alcohols with various molecular weights to
prepare double-emulsion core-shell nano-structures containing Doxorubicin

| Molecular | Core | Carrier | Doxo | Doxo loading |
|---|---|---|---|---|
| weight of | diameter | diameter | Encapsulation | capacity |
| PVA | (nm) | (nm) | efficiency (%)* | (mg/g carrier) |
| 16k | 108 | 158 | 18 | 0.23 |
| 47k | 47 | 144 | 9 | 0.12 |
| 61k | 31 | 137 | 6 | 0.08 |

*Encapsulation efficiency (%) = Doxo loading capacity/Doxo total amount × 100%

FIG. 10 is natural release curves of the double-emulsion core-shell nano-structures containing Doxorubicin. From FIG. 10, it can be known that when the molecular weight of the polyvinyl alcohol is greater and thus the oily shell is thicker, the natural release rate of the Doxorubicin from the double-emulsion core-shell nano-structures containing Doxorubicin is decreased.

### Embodiment 5

### Simultaneously Enclosing Doxorubicin and Paclitaxel by Single Emulsifying Method

Polyvinyl alcohols with various molecular weights were used to prepare double-emulsion core-shell nano-structures containing Doxorubicin and Paclitaxel by single emulsifying method. An aqueous solution and an organic solution were respectively prepared. The aqueous solution contained 0.02 g/ml 0.02 g/ml polyvinyl alcohol (MW 16,000, 47,000, and 61,000) and 0.002 g/ml Doxorubicin. The organic solvent used in the organic solution above was chloroform, and the organic solution contained 0.02 g/ml Paclitaxel and 0.02 g/ml IO-OA nanoparticles with diameters about 5 nm.

0.2 ml of the aqueous solution above and 0.5 ml of the organic solution above were mixed and then completely emulsified by 20 kHz supersonics. Next, the chloroform of the emulsion solution was completely volatized at room temperature. Finally, the product of the double-emulsion core-shell nano-structures were washed by deionized water and isolated by 8500 rpm centrifugation, and then re-dispersed in deionized water.

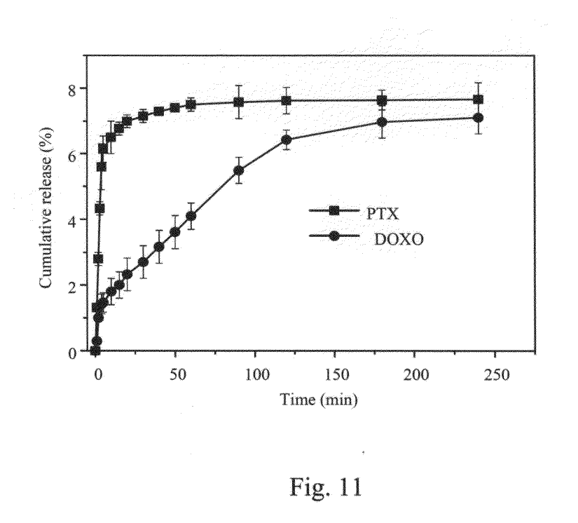

FIG. 11 is natural release curves of Doxorubicin and Paclitaxel from the double-emulsion core-shell nano-structures containing Paclitaxel and Doxorubicin, and the molecular weight of polyvinyl alcohol was 16,000. From FIG. 11, it can be known that the release curve of Doxorubicin from the double-emulsion core-shell nano-structures containing both Paclitaxel and Doxorubicin was similar to the release curve of Doxorubicin from the double-emulsion core-shell nano-structures containing only Doxorubicin. Similarly, the release curve of the Paclitaxel from the double-emulsion core-shell nano-structures containing both Paclitaxel and Doxorubicin was similar to the release curve of the Paclitaxel from the double-emulsion core-shell nano-structures containing only Paclitaxel. Accordingly, the release behavior is not significantly affected by containing both a hydrophilic drug and a hydrophobic drug.

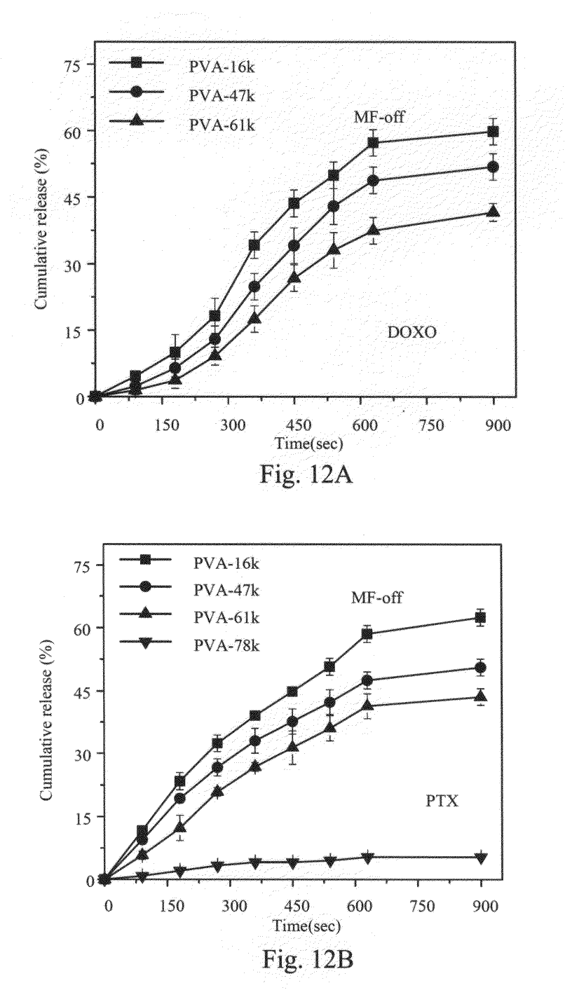

FIGS. 12A and 12B are controlled release curves of Doxorubicin and Paclitaxel from the double-emulsion core-shell nano-structures containing both hydrophobic Paclitaxel and hydrophilic Doxorubicin, respectively. From FIGS. 12A and 12B, it can be known that the controlled release rates of the Paclitaxel and Doxorubicin were slower when the molecular weight of the polyvinyl alcohol was greater. However, comparing with the natural release rates, the controlled release rates of the Paclitaxel and Doxorubicin were faster.

### Embodiment 6

### Simultaneously Enclosing Doxorubicin and Paclitaxel by Double Emulsifying Method

Double-emulsion core-shell nano-structures containing both hydrophobic Paclitaxel and hydrophilic Doxorubicin were prepared by following the double emulsifying method in FIG. 2B. In the first emulsifying step, a first aqueous solution and an organic solution were respectively prepared. The first aqueous solution above contained 0.02 g/ml polyvinyl alcohol (MW 16,000) and 0.002 g/ml Doxorubicin. The organic solvent used in the organic solution above was chloroform, and the organic solution contained 0.02 g/ml Paclitaxel and 0.02 g/ml IO-OA nanoparticles with diameters about 5 nm. 0.2 ml of the aqueous solution and 0.5 ml of the organic solution were mixed and then completely emulsified by 20 kHz supersonics to form water-in-oil emulsion solution.

Next, the second emulsifying step was performed. A second aqueous solution containing 0.02 g/ml polyvinyl alcohol (MW 16,000, 47,000, and 61,000) was prepared. The water-in-oil emulsion solution above and 1.75 ml of the second aqueous solution were mixed and then completely emulsified by 20 kHz supersonics to form a oil-in-water emulsion solution. Next, the chloroform of the oil-in-water emulsion solution was completely volatized at room temperature. Finally, the product of the double-emulsion core-shell nano-structures were washed by deionized water and isolated by 8500 rpm centrifugation, and then re-dispersed in deionized water.

The related data of the finally obtained double-emulsion core-shell nano-structures containing Doxorubicin are listed in Table 3. From Table 3, it can be known that when the molecular weight of the polyvinyl alcohol used in the second emulsifying step is greater, the volumes of the aqueous cores inside the double-emulsion core-shell nano-structures were smaller and thus the oily shells were thicker. Therefore, the encapsulation efficiency and the loading capacity of the hydrophilic Doxorubicin were decreased. However, comparing with the result of the single emulsifying method, the double emulsifying method obviously increased the encapsulation efficiency and the loading capacity of the Doxorubicin.

The molecular weight of the polyvinyl alcohol used in the second emulsifying step did not influence the encapsulation efficiency and the loading capacity of the hydrophobic Paclitaxel. Moreover, the difference of the encapsulation efficiency and the loading capacity between the single and the double emulsifying methods was not much.

TABLE 3
Double-emulsion core-shell nano-structures containing both
hydrophobic Paclitaxel and hydrophilic Doxorubicin was prepared by double
emulsifying method, and polyvinyl alcohols with various molecular weights were
used in the second emulsifying step.

| PVA MW | | Core | Carrier | Encapsulation | | Loading capacity | |
|---|---|---|---|---|---|---|---|
| First | Second | diameter | diameter | efficiency (%) | | (mg/g carrier) | |
| emulsifying | emulsifying | (nm) | (nm) | Doxo | PTX | Doxo | PTX |
| 16k | 16k | 105 | 155 | 41 | 96 | 0.53 | 9.12 |
| 16k | 47k | 50 | 148 | 32 | 96 | 0.42 | 9.12 |
| 16k | 61k | 34 | 140 | 24 | 97 | 0.31 | 9.21 |

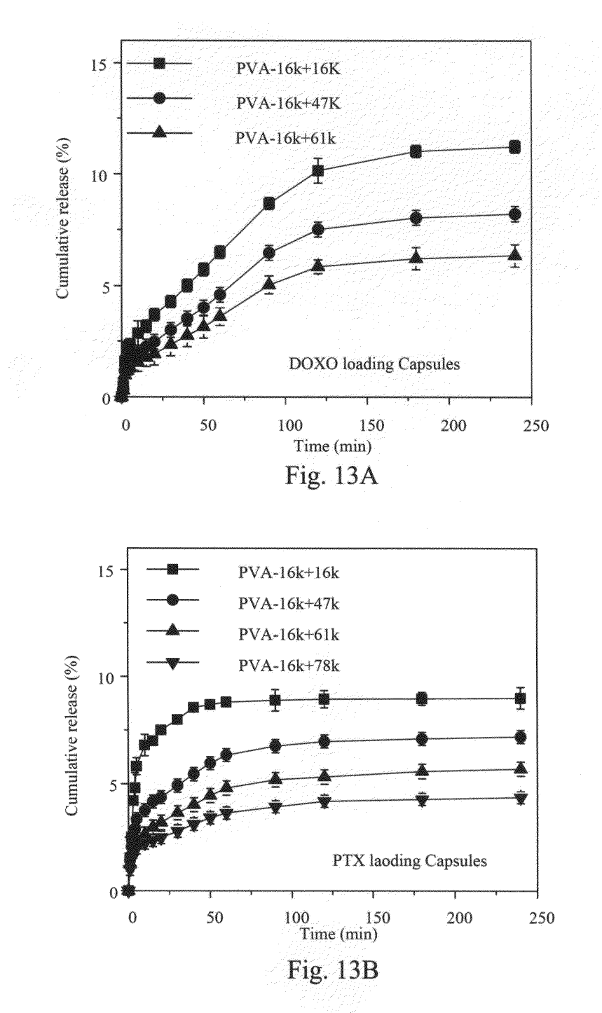

FIGS. 13A and 13B are natural release curves of Doxorubicin and Paclitaxel from the double-emulsion core-shell nano-structures containing both hydrophobic Paclitaxel and hydrophilic Doxorubicin, respectively. From FIGS. 13A and 13B, it can be known that the greater of the molecular weights of the polyvinyl alcohol was, the slower the natural release rates of the Paclitaxel and Doxorubicin were, since the thickness of the oily shells was increased.

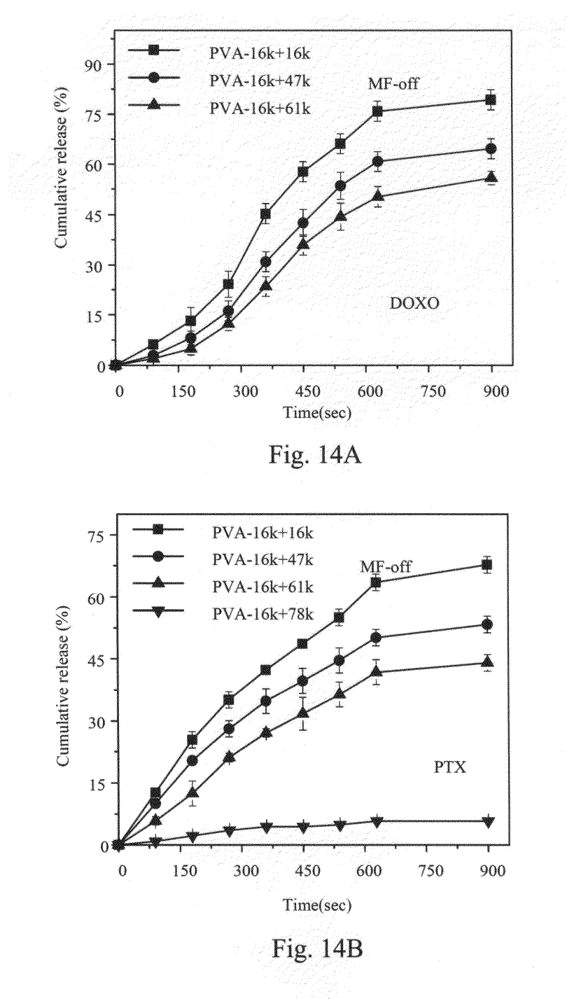

FIGS. 14A and 14B are controlled release curves of Doxorubicin and Paclitaxel from the double-emulsion core-shell nano-structures containing both hydrophobic Paclitaxel and hydrophilic Doxorubicin, respectively. From FIGS. 14A and 14B, it can be known that the release rate of the Paclitaxel and Doxorubicin can be increased by applying an alternative magnetic field. Similarly, the release rates of the Paclitaxel and Doxorubicin were decreased when the molecular weight of the polyvinyl alcohol used in the second emulsifying step was increased.

### Embodiment 7

### Preparing Double-Emulsion Core-Shell Nano-Structures by PVP

Following the single emulsifying method of FIG. 2A, double-emulsion core-shell nano-structures were prepared by polyvinyl pyrrolidone. An aqueous solution of 0.8 g/ml polyvinyl pyrrolidone (MW 800,000) was prepared. An organic solution contained 0.02 mg/ml IO-OA nanoparticles was then prepared. The organic solvent used was chloroform.

1 ml of the aqueous solution above and 0.4 ml of the organic solution above were mixed, and then completely emulsified by 20 kHz supersonics. Next, the chloroform of the emulsion solution was completely volatized at room temperature. Finally, the product of the double-emulsion core-shell nano-structures were washed by deionized water and isolated by 8500 rpm centrifugation, and then re-dispersed in deionized water.

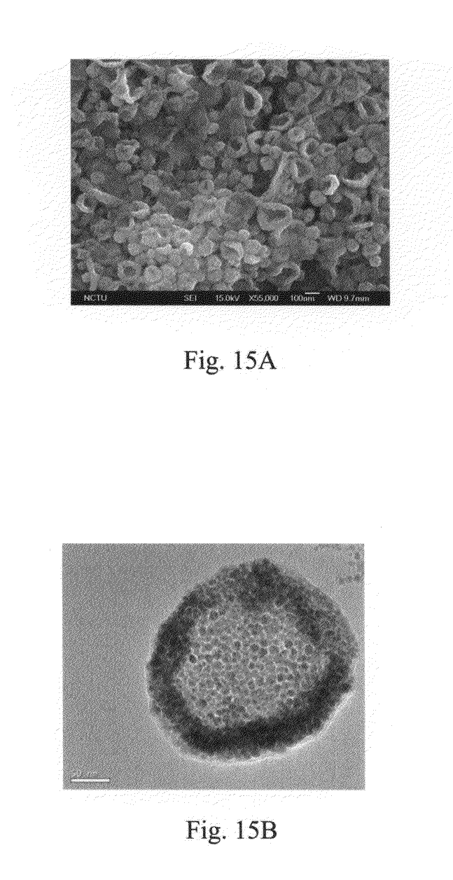

FIGS. 15A and 15B are SEM and TEM images of the obtained double-emulsion core-shell nano-structures made by PVP. Similar to the Embodiment 1, since the double-emulsion core-shell nano-structures had been vacuum-dried and the polymer chain of polyvinyl pyrrolidone is soft, the oily shells were collapsed in a bowl shape. In FIG. 15B, it can be seen that the double-emulsion core-shell nano-structures were in a hollow sphere structure and IO-OA nanoparticles (darker color ones) were dispersed in the oily shells.

### Embodiment 8

### Cytocompatibility Tests

Since the various double-emulsion core-shell nano-structures above can be drug nano-carriers to increase the transportation efficiency and absorptivity of drugs, and the total usage amount of the drugs can be decreased. Therefore, some functional groups, such as carboxylic groups, aldehyde groups, amino groups, hydroxyl groups, amide groups, or sulfonyl groups, can be used to modify the surfaces of the double-emulsion core-shell nano-structures. Then, the surface-modified double-emulsion core-shell nano-structures can bind to various antibodies or peptides to be encapsulated by certain types of cell and act as a targeted drug.

The test method of the cytocompatibility test is described below. Double-emulsion core-shell nano-structures with and without surface modification were prepared first. The above double-emulsion core-shell nano-structures contained red-light quantum dots. Next, the double-emulsion core-shell nano-structures with and without surface modification were respectively contact with breast cancer cells MCF-7 for a period of time and then observed by fluorescence microscope.

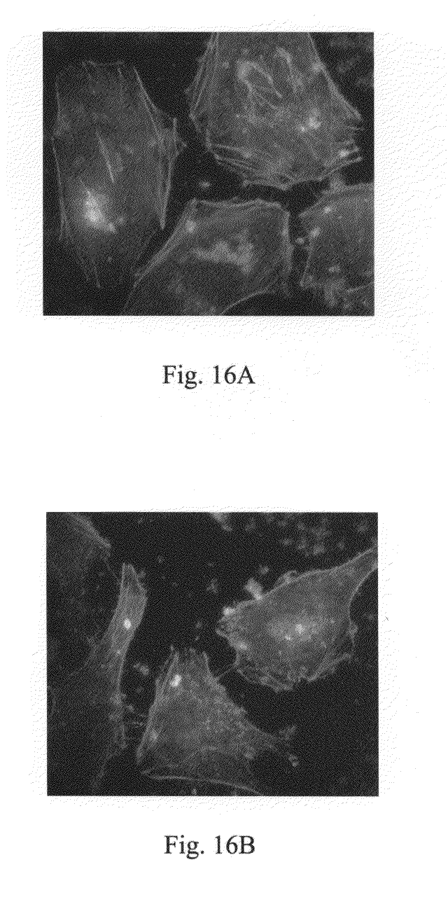

FIGS. 16A and 16B are the cytocompatibility test results of the double-emulsion core-shell nano-structures with and without surface modification, respectively. In FIGS. 16A and 16B, the red color represents the red-light quantum dots, the blue color represents the cell nuclei, and the green color represents the cytoplast. In FIG. 16A, since the double-emulsion core-shell nano-structures were not surface modified, and thus could not be easily swallowed by the breast cancer cells MCF-7. However in FIG. 16B, since the double-emulsion core-shell nano-structures were surface modified by carboxylic groups, and thus could be easily swallowed by the breast cancer cells MCF-7.

From the disclosure above, it can be known that a water-soluble polymer with various molecular weights can be used to prepare double-emulsion core-shell nano-structures having aqueous cores and oily shells by single or double emulsifying methods. The obtained double-emulsion core-shell nano-structures can be used as drug carriers to enclose hydrophilic and hydrophobic drugs. If the double-emulsion core-shell nano-structures are surface modified, the double-emulsion core-shell nano-structures can also be carriers for targeted drugs. Moreover, the paramagnetic nanoparticles and an alternative magnetic field can be used to locally heat the double-emulsion core-shell nano-structures to controlled release the drugs in the double-emulsion core-shell nano-structures.

All the features disclosed in this specification (including any accompanying claims, abstract, and drawings) may be replaced by alternative features serving the same, equivalent or similar purpose, unless expressly stated otherwise. Thus, each feature disclosed is one example only of a generic series of equivalent or similar features.

What is claimed is:

1. A double-emulsion core-shell nano-structure, comprising:
an aqueous core; and
an oily shell surrounding the aqueous core, wherein a composition of the oily shell comprises a water-soluble polymer and a plurality of hydrophobic paramagnetic nanoparticles, without using a surfactant.

2. The double-emulsion core-shell nano-structure of claim 1, wherein the water-soluble polymer is polyvinyl alcohol having a molecular weight of 3,000-130,000 or polyvinyl pyrrolidone having having a molecular weight of 400,000-2,800,000.

3. The double-emulsion core-shell nano-structure of claim 1, wherein the hydrophobic paramagnetic nanoparticles are Fe₂O₃, Fe₃O₄, CoFe₂O₄ or MnFe₂O₄ nanoparticles, which are modified by hydrophobic functional groups thereon.

4. The double-emulsion core-shell nano-structure of claim 1, wherein the oily shell comprises a hydrophobic drug.

5. The double-emulsion core-shell nano-structure of claim 1, wherein the aqueous shell comprises a hydrophilic drug.

6. A method of preparing a double-emulsion core-shell nano-structure, the method comprising:
preparing an aqueous solution comprising a water-soluble polymer;
preparing an organic solution comprising a plurality of hydrophobic paramagnetic nanoparticles; and
mixing and stirring the aqueous solution and the organic solution to form a plurality of double-emulsion core-shell nano-structures in an emulsion solution, wherein each of the double-emulsion core-shell nano-structures comprises an aqueous core and an oily shell surrounding the aqueous core, and the oily shell comprises the water-soluble polymer and the hydrophobic paramagnetic nanoparticles.

7. The method of claim 6, wherein the organic solution comprises a hydrophobic drug.

8. The method of claim 6, wherein the aqueous solution comprises a hydrophilic drug.

9. The method of claim 6, wherein the water-soluble polymer is polyvinyl alcohol having a molecular weight of 3,000-130,000 or polyvinyl pyrrolidone having a molecular weight of 400,000-2,800,000.

10. The method of claim 6, further comprising removing an organic solvent of the emulsion solution.

11. The method of claim 6, wherein the hydrophobic paramagnetic nanoparticles are Fe₂O₃, Fe₃O₄, CoFe₂O₄ or MnFe₂O₄ nanoparticles having hydrophobic functional groups on surfaces thereof.

12. The method of claim 6, wherein a method of the mixing and stirring step comprises ultrasound sonication.

13. A method of preparing a double-emulsion core-shell nano-structure, the method comprising:
preparing a first aqueous solution comprising a hydrophilic drug and a first water-soluble polymer;
preparing an organic solution comprising a plurality of hydrophobic paramagnetic nanoparticles; and
mixing and stirring the first aqueous solution and the organic solution to form a water-in-oil emulsion solution;
preparing a second aqueous solution comprising a second water-soluble polymer;
mixing and stirring the water-in-oil emulsion solution and the second aqueous solution to form a oil-in-water emulsion solution containing a plurality of double-emulsion core-shell nano-structures, wherein each of the double-emulsion core-shell nano-structures comprises an aqueous core and an oily shell surrounding the aqueous core, the aqueous core comprises the hydrophilic drug and the oily shell comprises the first and the second water-soluble polymers and the hydrophobic paramagnetic nanoparticles.

14. The method of claim 13, wherein the organic solution comprises a hydrophobic drug.

15. The method of claim 13, wherein the kinds and molecular weights of the first and the second water-soluble polymers are different.

16. The method of claim 13, further comprising removing an organic solvent of the oil-in-water emulsion solution.

17. The method of claim 13, wherein the first and the second water-soluble polymers are polyvinyl alcohol having a molecular weight of 3,000-130,000 or polyvinyl pyrrolidone having a molecular weight of 400,000-2,800,000.

18. The method of claim 13, wherein the hydrophobic paramagnetic nanoparticles are Fe₂O₃, Fe₃O₄, CoFe₂O₄ or MnFe₂O₄ nanoparticles having hydrophobic functional groups on surfaces thereof.

19. The method of claim 13, wherein a method of the mixing and stirring step comprises ultrasound sonication.

20. A method of controlled releasing a drug, the method comprising:
enclosing a drug in the double-emulsion core-shell nano-structure of claim 1, wherein the drug is enclosed in the aqueous core when the drug is hydrophilic, and the drug is enclosed in the oily shell when the drug is hydrophobic; and
increasing the releasing rate of the drug by turning on an alternative magnetic field and decreasing the releasing rate of the drug by turning off the alternative magnetic field.
